(19) United States
(12) Patent Application Publication
(10) Pub. No.: US 2011/0098517 A1
(43) Pub. Date: Apr. 28, 2011

(54) Use of Subfluorinated Carbons as a Solid Lubricant

(76) Inventors: André Hamwi, Clermont-Ferrand (FR); Marc Dubois, Clermont-Ferrand (FR); Katia Guerin, Du Chateau (FR); Rachid Yazami, Etats-Unisdamerique (FR)

(21) Appl. No.: 12/995,902

(22) PCT Filed: May 27, 2009
(86) PCT No.: PCT/FR09/00613

§ 371 Date: Jan. 5, 2011

(30) Foreign Application Priority Data
Jun. 3, 2008 (FR) 08/03047

(51) Int. Cl. C07C 23/46; B82Y 30/00

(52) U.S. Cl. 570/130; 977/734; 977/902

(57) ABSTRACT

The invention relates to the use of subfluorinated carbons as a solid lubricant. Said subfluorinated carbons simultaneously contain fluorinated carbon domains with a (CF)n structure and non-fluorinated graphitic carbon domains, in powder form, as a solid lubricant. The invention can be used in the field of solid lubricants.

The invention relates to the use of subfluorinated carbons as a solid lubricant.

Graphitic carbon is known to be a solid lubricant.

However, graphitic carbon can only be used as a solid lubricant in a humid atmosphere and not in ambient air, that is with a relative humidity of about 55%.

It has therefore been proposed to use (CFₓ)ₙ type graphite fluorides as a solid lubricant.

These graphite fluorides can be used in various atmospheres, that is humid air, dry air, dry argon, and up to temperatures of 550° C. They can also be used under vacuum space, that is under ultrahigh vacuum of 10⁻⁸ to 10⁻⁹ torr, while providing a low wear rate.

These graphite fluorides can be obtained by various methods. The first is a method of direct fluorination of graphite at temperatures between 420° C. and 550° C. Such a method is described in W. Rudorff et al., Z. Anorg. Allgem. Chem., 253, 281 (1947). The graphite fluorides thus obtained at higher temperature, in particular at 550° C., correspond to a (CF)ₙ structure in which the carbon layers consist of an infinite network of hexagonal rings having a chair or boat shape, bonded together by covalent bonds formed between the sp³ carbon atoms. Each carbon atom is also bonded to a fluorine atom by a covalent bond.

Another method of synthesis by direct fluorination is described by Y. Kita et al., in J. Am. Chem. Soc., 101, 3832 (1979). This method yields a graphite fluoride having the formula (C₂F)ₙ.

Graphite fluorides which are carbon-fluorine inclusion complexes are also known. These graphite fluorides have been obtained by various carbon fluorination methods at ambient temperature. At ambient temperature, fluorine, if used alone, does not react with graphite. In some of these methods, graphite is reacted with a F₂+HF gas mixture in the presence or absence of a metal fluoride such as LiF, SbF₅, WF₆, CuF₂, AgF or IF₅, followed by post-heat treatment in F₂ gas at temperatures between 100° C. and 600° C.

The family of subfluorinated carbons is also known.

The main feature of this carbon family called subfluorinated carbons is the presence of nonfluorinated graphitic carbon domains intimately mixed with fluorinated carbon domains having a (CF)ₙ structure.

Preferably, the nonfluorinated graphitic carbon domains are nanodomains.

In the context of the present invention, nanodomains means domains whereof at least one dimension is between 1 nanometer and 1 micron inclusive, preferably between 1 and 300 nanometers inclusive.

These are the subfluorinated carbons used in the invention as a solid lubricant.

In fact, these subfluorinated carbons have an excellent friction coefficient lower than 0.1 at 25° C., and in ambient air, that is with a relative humidity of about 55%, and even after 100 cycles, that is 100 return trips of the friction ball on the sample, as described below.

Thus, the invention proposes the use of subfluorinated carbons comprising domains of purely graphitic carbon, that is nonfluorinated, preferably having at least one dimension of between 1 nanometer and 1 micron, more preferably between 1 and 300 nanometers, in combination with fluorinated carbon domains having a (CF)ₙ structure, in powder form, as a solid lubricant.

Preferably, the molar percentage of graphitic carbon compared to the total number of moles of subfluorinated carbon is 5% or more but strictly lower than 100%.

Also preferably, the subfluorinated carbon is obtained by fluorinating a carbon matrix at a temperature between 300° C. and 500° C. inclusive.

Preferably, this carbon matrix has a graphitic structure.

In a first preferred embodiment of the invention, the carbon matrix having a graphitic structure consists of nanofibers and/or nanotubes and/or nanocones and/or nanodisks and/or nanoparticles of graphitic carbon.

More preferably, in this first preferred embodiment of the invention, the carbon matrix consists of graphitic carbon nanofibers and said matrix is fluorinated by direct fluorination at a temperature between 370° C. and 500° C. inclusive.

Even more preferably, in this first preferred embodiment of the invention, the carbon matrix consists of graphitic carbon nanofibers and is fluorinated by direct fluorination at a temperature between 400° C. and 425° C. inclusive.

In a second preferred embodiment of the invention, the carbon matrix consists of carbon and/or coke and/or petroleum pitch having a graphitic structure.

The invention will be better understood and other advantages and features thereof will appear more clearly from a reading of the explanatory description that follows, with reference to the appended figures in which:

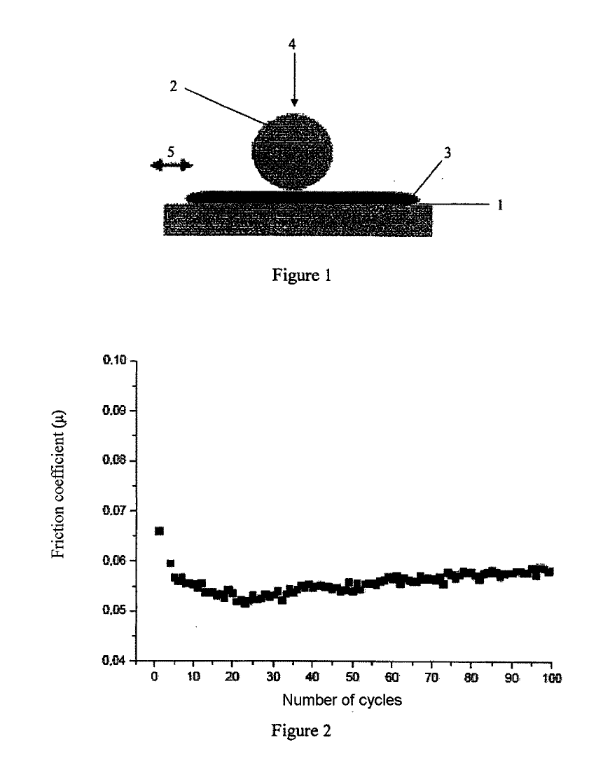

FIG. 1 schematically shows the apparatus used for the friction tests in order to determine the friction coefficient of the various fluorinated and subfluorinated carbons tested;

FIG. 2 shows the variation in the friction coefficient of a subfluorinated carbon of the invention as a function of the number of friction cycles;

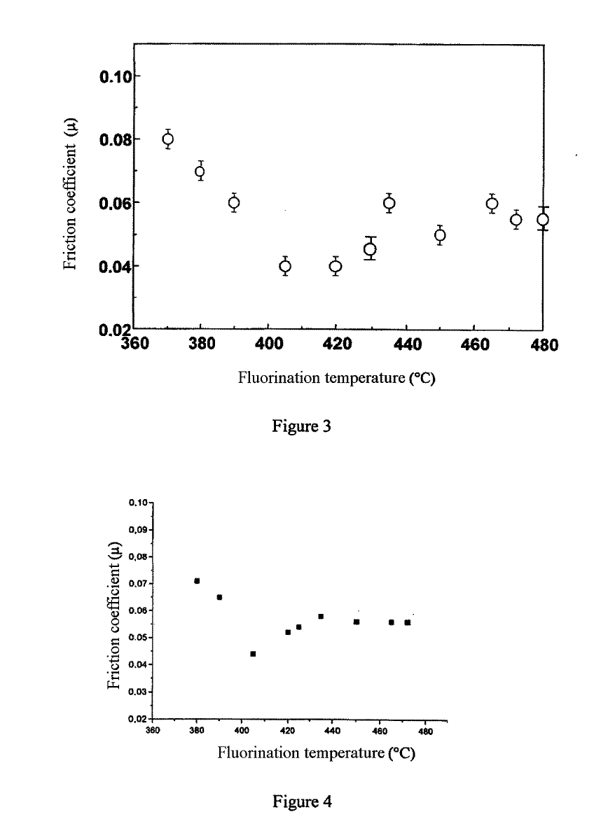

FIG. 3 shows the friction coefficients of various subfluorinated carbons of the invention after 60 friction cycles;

FIG. 4 shows the friction coefficients of various subfluorinated carbons of the invention after 100 friction cycles;

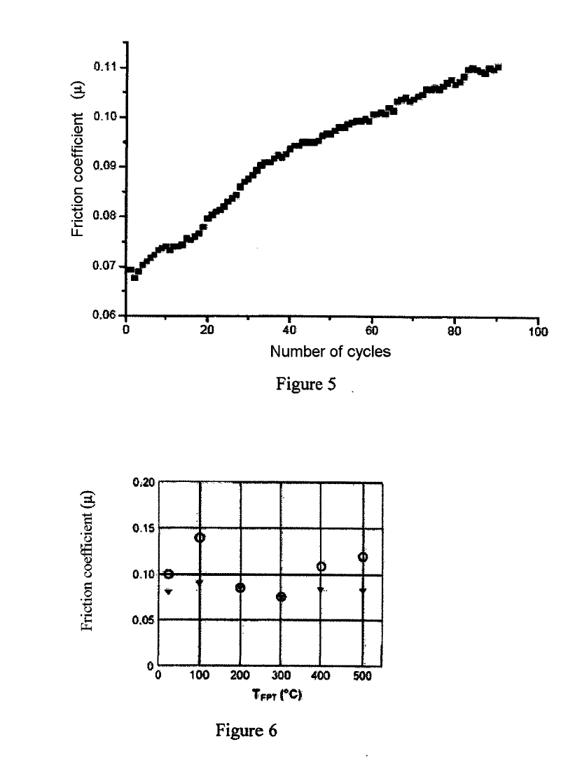

FIG. 5 shows the variation in the friction coefficient of a graphite fluoride of the prior art having the formula CF_{1.1} as a function of the number of friction cycles; and

FIG. 6 shows the friction coefficients of various graphite fluorides having undergone a post-heat treatment at temperatures of 100° C., 200° C., 400° C., 500° C. and without post-heat treatment in F₂, after four friction cycles and after sixty friction cycles.

The subfluorinated carbons used in the invention are obtained from various carbon matrices.

They can be obtained from a graphitic carbon matrix, which may consist of a powder whereof the grains are larger than one micron, on average, or of nanomaterials, that is nanofibers and/or nanotubes, and/or nanodisks, and/or nanocones, and/or nanoparticles of graphitic carbon.

Patent application WO 97/41061 in particular describes a method for obtaining subfluorinated carbons from a carbon matrix consisting of a graphitic carbon powder whereof the grains are larger than one micron, on average.

According to the method described in WO 97/41061, in a first step, the carbon matrix consisting of graphite or graphitizable carbon having a mosaic texture, is reacted with a HF+F₂ gas mixture in the presence of a fluoride MFₙ at a temperature between 15° C. and 80° C., where M is the element selected from I, Cl, Br, Re, W, Mo, Nb, Ta, B, Ti, P, As, Sb, S, Se, Te, Pt, Ir and Os and n is the valency of the element M, with n≦7. In a second step, the compound obtained at the end of the first step is reacted with fluorine for 1 to 20 hours at a temperature between 20° C. and 400° C.

Further details about this synthesis method are provided in patent application WO 97/41061.

The subfluorinated carbons of the invention can also be obtained by direct fluorination of graphitic carbon nanomaterials. In the context of the present invention, nanomaterials means nanofibers, nanotubes, nanodisks, nanocones, nanoparticles or mixtures thereof.

A method for obtaining subfluorinated carbons from graphitic carbon nanomaterials is described in patent application WO 2007/126436.

According to the method described in WO 2007/126436, the graphitic carbon nanomaterials are subjected to a gas source of elemental fluorine, under a pressure between 1 atmosphere and 0.1 atmosphere, at a temperature between 375° C. and 480° C. inclusive, for a predefined time according to the mass of carbon and the fluorine flow rate.

The nanomaterials thus obtained have an F/C atomic ratio which may be higher than 1, measured by NMR of fluorine 19.

Thus, the subfluorinated carbons used in the invention may have a total F/C atomic ratio higher than 1.

In fact, what characterizes the subfluorinated carbons used as a solid lubricant in the invention is the fact that they comprise nonfluorinated graphitic carbon domains intimately mixed with fluorinated carbon domains. In fact, at the periphery of the purely graphitic carbon domains or fluorinated carbon domains, zones exist in which the fluorine content is higher.

The subfluorinated carbons of the invention can be synthesized, as already stated, from graphitic carbon nanofibers, by direct fluorination, with molecular fluorine at temperatures higher than 300° C., preferably between 300° C. and 500° C. inclusive.

To keep the nonfluorinated graphitic carbon domains intimately mixed with fluorinated carbon domains, and because of the very high reactivity of molecular fluorine, severe control of the production conditions is necessary. This control can be achieved by limiting the reaction temperature or time or by diluting the molecular fluorine with nitrogen or argon, or by the application of a suitable gas flow rate. Once the conditions are set, the total fluorination rate of the subfluorinated carbon obtained is controlled by controlling the weight gain: the accommodation of one atom of fluorine leads to a weight gain of 19 g per mole of carbon.

Another method for producing the subfluorinated carbons of the invention, in particular by fluorinating graphitic carbon nanofibers, consists in employing a fluorinating agent rather than molecular fluorine. This fluorinating agent is a fluoride of an element that may have a number of oxidation states, such as terbium, which exists in the form of Tb³⁺ and Tb⁴⁺ ions.

The thermal decomposition of this fluorinating agent, for example between 200° C. and 450° C. for TbF₄, generates TbF₃ and either atomic or molecular fluorine, which can then react with the carbon material at the target temperature (300° C.<T<500° C.), while the decomposition temperatures of the fluorinating agent and of the carbon may be different. In this case, the quantity of fluorine that has reacted is controlled by the quantity of fluorinating agent. An excess of fluorinating agent is applied. For example, to obtain an F/C ratio of 1, the number of moles of TbF₄ is 1.5 per mole of C. Further details about this method are given in “Fluorination of poly(p-phenylene) using TbF₄ as fluorinating agent”, W. Zhang et al., Journal of Fluorine Chemistry, 128 (2007) 1402-1409.

The subfluorinated carbons of the invention can also be obtained from carbon nanomaterials not initially having a graphitic structure, but which consist of a graphitizable carbon material. The method for synthesizing such subfluorinated carbons is described in patent application WO 2007/126436.

Thus, the subfluorinated carbons of the invention can be prepared from various initial carbons, that is from carbon, coke, petroleum pitch, nanotubes, nanofibers, nanodisks, nanocones, nanoparticles of carbon that either have a graphitic structure or are graphitizable.

The chemical composition of the subfluorinated carbons used in the invention, that is the atomic ratio of fluorine “x” in CFₓ, can be determined by two methods: by weight gain and by NMR of fluorine 19 in comparison with a calibration sample of polytetrafluoroethylene (PTFE).

Good agreement between the two methods is obtained, except for the high fluorination temperatures, that is temperatures higher than 465° C., because of the formation of perfluorinated and volatile alkyls (CF₄, C₂F₆, etc.).

For this reason, in the examples that follow, the fluorine content indicated is the fluorine content measured by quantitative fluorine-19 NMR, and the F/C ratio is calculated according to the fluorine content thus calculated. However, this method becomes inaccurate when the F/C ratios are low, that is lower than 0.04. This is why, in Table 1 below, the F/C ratios lower than 0.06 are indicated as approximate values.

The subfluorinated carbons used in the invention were characterized by X-ray diffraction, FTIR spectroscopy and Raman spectroscopy, high-resolution solid-state NMR (¹⁹F and ¹³C) and electron paramagnetic resonance (EPR).

The percentage, in moles, of nonfluorinated carbon, was measured by the deconvolution of the ¹³C NMR spectra. The signal of the nonfluorinated carbons is observed at 120 ppm/TMS as for pure graphite. The percentage of graphitic carbon is obtained by determining the ratio of the peak areas S_{Cgraphitic}/(S_{Cgraphitic}+S_{C-F}+S_{C-F}+S_{C-C}).

In this equation, S_{Cgraphitic} is the area of the signal of graphitic carbon, S_{C-F} is the area of the signal of the carbons bonded by covalent C—F bonds, S_{C-F} is the area of the signal of the carbons bonded by semi-covalent C—F bonds (carbon sp² in weak interaction with the fluorine atoms) and S_{C-C} is the area of the signal of the diamond type carbons.

However, for a purely graphitic carbon content higher than 90%, the measurement becomes inaccurate due to the error margins inherent in this method. For this reason, in the examples that follow, when the nonfluorinated graphitic carbon content is higher than 90%, it is only indicated as above 90%.

However, the subfluorinated carbons of the invention always contain strictly less than 100% of graphitic carbon because they will have been fluorinated.

More precisely, the subfluorinated carbons used in the invention contain at least 5%, but less than 100% of graphitic carbon.

For a better understanding of the invention, several embodiments and implementations thereof are now described.

These examples are given purely for illustration, and must not in any case be considered as limiting the invention.

### EXAMPLE 1

### Synthesis of Subfluorinated Carbons From a Matrix Consisting of Graphitic Carbon Nanofibers by the Method of Direct Fluorination With Molecular Fluorine

The carbon matrix is weighed to a mass of about 20 g.

The carbon matrix is previously degassed under a rough vacuum for two hours. It is then introduced into a cylindrical nickel reactor having a volume of 4 liters. Flushing with N₂ is carried out for two hours at a temperature of 200° C., and the temperature is then increased with a temperature ramp of 5° C.min⁻¹ to the desired fluorination temperature. Once the desired temperature is reached, a stream of molecular fluorine (about 2 g per hour) is applied at ambient pressure for a period of about 16 hours, which varies according to the desired fluorine content.

The subfluorinated carbon obtained is then allowed to cool to ambient temperature and its chemical composition, that is the atomic percentage of fluorine in the subfluorinated carbon, is determined by the fluorine-19 NMR method described above.

The percentage of nonfluorinated graphitic carbon in the products obtained was calculated as previously described by deconvolution of the ¹³C NMR spectra of these products.

The reaction temperatures with fluorine, the fluorination times, the atomic F/C ratio and the percentage of graphitic carbon measured on the samples obtained in this example are given in Table 1 below.

In Table 1, the samples prepared in this example are denoted “CNF” followed by the reaction temperature with the fluorine. More precisely, the samples obtained in this example are denoted “CNF-370” to “CNF-480”.

### EXAMPLE 2

### Synthesis of Subfluorinated Carbons by Direct Fluorination by Molecular Fluorine of a Carbon Matrix Consisting of Graphitic Carbon Particles Larger Than One Micron

A graphitic carbon powder having an average grain size of 30 μm is weighed to a mass of about 20 g. This carbon matrix is treated and analyzed as in example 1.

The fluorination temperature, the atomic F/C ratio and the molar percentage of nonfluorinated graphitic carbon present in the samples obtained are given in Table 1.

In Table 1, the samples obtained in this example are denoted “graphite” followed by the fluorination temperature used.

### EXAMPLE 3

### Synthesis of Subfluorinated Carbons by a Fluorinating Agent from Graphitic Carbon Nanofibers

The carbon matrix consisting of graphitic carbon nanofibers is weighed to a mass of about 60 mg. It is then introduced, using a nickel boat, into a cylindrical nickel reactor having a volume of 0.7 liter, at the same time as 1.175 g of TbF₄ in a second nickel boat. The boat containing the TbF₄ is positioned in the zone 1 of the two-zone furnace, while the boat containing the carbon matrix is positioned in the furnace temperature zone corresponding to the desired fluorination temperature. A rough vacuum is then applied to the reactor (10⁻² atm). The furnace temperature is set at 500° C. to promote the decomposition of the TbF₄ to TbF₃ and the liberation of atomic and/or molecular fluorine which then reacts for 16 hours with the carbon matrix, which is heated between 300 and 500° C. inclusive. To reach the temperature setpoint, a temperature ramp of 5° C./min is applied.

The subfluorinated carbon obtained is then allowed to cool to ambient temperature and analyzed as in example 1.

Table 1 shows the fluorination temperature, the fluorination time, and the F/C atomic ratio and molar percentage of nonfluorinated graphitic carbon in the samples obtained by this method.

In Table 1, the samples obtained in this example are denoted “CNF-C” followed by the indication of the fluorination temperature.

TABLE 1

| | Reaction | | | % |
|---|---|---|---|---|
| | temperature | Time | Atomic | graphitic |
| Sample | (° C.) | (h) | F/C | C |
| CNF-370 | 370 | 16 | ~0.04 | >90 |
| CNF-380 | 380 | 16 | 0.06 | >90 |
| CNF-390 | 390 | 16 | 0.09 | >90 |
| CNF-405 | 405 | 16 | 0.15 | >90- |
| CNF-420 | 420 | 16 | 0.39 | 82 |
| CNF-428 | 428 | 16 | 0.59 | 25 |
| CNF-435 | 435 | 16 | 0.68 | 25 |
| CNF-450 | 450 | 16 | 0.74 | 20 |
| CNF-465 | 465 | 16 | 0.77 | 12.7 |
| CNF-472 | 472 | 16 | 0.90 | 13 |
| CNF-480 | 480 | 16 | 1.04 | 7 |
| Graphite-350 | 350 | 12 | 0.51 | 19 |
| Graphite-380 | 380 | 12 | 0.60 | 8 |
| CNF-C420 | 420 | 13 | 0.12 | 87 |
| CNF-C450 | 450 | 13 | 0.56 | 46 |
| CNF-C480 | 480 | 13 | 0.70 | 35 |
| CNF-C500 | 500 | 13 | 0.91 | 20 |

### EXAMPLE 4

### Physical and Chemical Characterizations of Subfluorinated Carbons Obtained in Examples 1 to 3

The subfluorinated carbons obtained in examples 1 to 3 were characterized by X-ray diffraction, FTIR spectroscopy and Raman spectroscopy, solid-state high-resolution NMR (¹⁹F and ¹³C) and electron paramagnetic resonance (EPR).

The ¹⁹F NMR shows that the C—F bond in the subfluorinated carbons used in the invention is covalent. The ¹³C NMR shows the presence of graphitic carbons C sp2 (hence nonfluorinated), carbons strongly bonded to the fluorine (covalent bond) C sp3, carbons more weakly bonded to the fluorine C sp2 and diamond carbons C sp3.

### EXAMPLE 5

### Friction Test for the Sample Denoted CNF-435 in Table 1

The tribological parameters were determined using an alternating sphere on plane tribometer shown schematically in FIG. 1.

As shown in FIG. 1, this tribometer comprises a 100C6 steel plane, denoted 1 in FIG. 1, measuring 10×2 mm, and a ball, denoted 2 in FIG. 1, having a diameter of 10 mm and also made from 100C6 steel. Force sensors, not shown in FIG. 1, are connected to a data acquisition system that serves to monitor the experiments from a computer.

The method for depositing the lubricant film, here the sample denoted CNF-435 in Table 1, is called burnishing.

In this method, the sample to be tested is spread in powder form on a plane and crushed using another plane, followed by removal of the surplus. The planes used are first polished using sandpaper (1000 μm and 400 μm) to ensure good adhesion of the lubricant film. The surface irregularities are estimated at 100 nm peak-to-peak.

The planes are then subjected to ultrasound in baths of ethanol and acetone to remove the impurities and abrasive particles.

The sample denoted CNF-435 is then deposited on a plane thereby forming the lubricant film, denoted 3 in FIG. 1.

The test consists in applying a normal force Fn, denoted 4 in FIG. 1, on a steel ball, denoted 2 in FIG. 1, and imposing an alternating movement thereon, denoted 5 in FIG. 1, allowing measurement of the tangential force Ft.

The macroscopic friction coefficient p is obtained by calculating the ratio of the tangential force measured in the test to the normal force applied:

μ=Ft/Fn

During the test, a normal load of 10 N (weight of about 1 kg) is applied, giving rise to a contact diameter of 86 μm (Hertz theory) and a pressure of 0.65 GPa. The speed of movement of the ball on the plane is constant and is 2 mm per second. The tests are performed at 25° C. in ambient air (relative humidity higher than 55%). Several plots are made on different portions of the film in order to determine its intrinsic tribological properties, independent of the deposit. The study consisted in tracking the variation in the friction coefficient of the material to be tested as a function of the number of cycles, which varies between 0 and 100. The number of cycles is the number of return trips of the ball on the sample. These tests were performed in ambient air.

FIG. 2 shows the variation in the friction coefficient for the sample denoted CNF-435 in Table 1. This subfluorinated carbon was obtained by fluorinating graphitic carbon nanofibers at 435° C. This variation is representative of the variation in the friction coefficient of the fluorinated carbon nanofibers for fluorination rates of between ˜0.04 and 1.1 inclusive.

As shown in FIG. 2, the subfluorinated carbons of the invention are excellent solid lubricants, because their friction coefficient is lower than 0.1.

### EXAMPLE 6

### Friction Tests on the Products Obtained in Example 1

The same tests as in example 5 were performed on the other products obtained in example 1.

FIG. 3 shows the variation in the friction coefficient μ for the 60th cycle for the various subfluorinated carbons obtained in example 1.

As shown in FIG. 3, even after 60 friction cycles, the friction coefficient of the subfluorinated carbons used in the invention remains lower than 0.1.

FIG. 4 shows the variation in the friction coefficient μ for the 100th cycle for the various subfluorinated carbons obtained in example 1.

As shown in FIG. 4, even after 100 friction cycles, the friction coefficient of the subfluorinated carbons clearly remains lower than 0.1.

For comparison, the initial value of the friction coefficient of nonfluorinated carbon nanofibers is 0.12.

### COMPARATIVE EXAMPLE 1

The tribological behavior of a carbon fluoride of the prior art obtained at high temperature was tested in the same way as described in example 3.

The carbon fluoride of the prior art used has a composition CF_{1.1}. It was obtained by direct fluorination at 600° C. for 5 hours of a graphitic carbon, natural graphite, having an average grain size of 6 microns (UF₄ supplied by Carbone Lorraine). This carbon did not contain any nonfluorinated graphitic carbon domains.

FIG. 5 shows the variation in the friction coefficient of the carbon fluoride of the prior art as a function of the number of cycles.

As shown in FIG. 5, for the first four cycles, the friction coefficient is 0.07 and then gradually increases during the friction test. At 60 cycles, the friction coefficient reaches the value of 0.10.

For comparison, the friction coefficient for the 60th cycle of the subfluorinated carbons used in the invention remains lower than or equal to 0.08.

Thus, the subfluorinated carbons of the invention have exceptional and durable lubricating properties, in comparison with pure graphite and in comparison with the carbon fluorides of the prior art.

However, a fluorination temperature zone of the subfluorinated carbons used in the invention exists, in which the tribological performance is particularly advantageous, that is, fluorination temperatures between 405° C. and 420° C.

In fact, the subfluorinated carbons used in the invention obtained at these temperatures have a lower fluorine content, but the fluorine atoms present are organized in the carbon matrix with a fluorine content between 0.1 and 0.5.

### COMPARATIVE EXAMPLE 2

Graphite fluorides of the prior art were synthesized by fluorination at ambient temperature of natural Madagascar graphite with a mixture of HF, F₂ and IF₅. The chemical composition of the product obtained is CF_{0.73} (IF₅)_{0.02}(HF)_{0.06}. A post-heat treatment is then carried out in fluorine gas at temperatures between 100° C. and 600° C. inclusive.

The compounds are denoted T_{FPT} where FPT is the post-heat treatment temperature.

The samples obtained were tested for their tribological properties, as described in example 6.

The friction coefficients of these prior art materials, after four cycles and one hundred cycles, are shown in FIG. 6.

The friction coefficients after four cycles are indicated by solid inverted triangles in FIG. 6, while the friction coefficients after one hundred cycles are indicated by circles in FIG. 6.

In FIG. 6, the friction coefficients, after three cycles, of the prior art graphite fluorides synthesized in this example, are shown by solid inverted triangles, and those after 100 cycles are shown by circles.

As may be observed in FIG. 6, after three friction cycles, all the graphite fluorides obtained in this example have a friction coefficient in the interval from 0.07 to 0.09.

However, after one hundred cycles, the friction coefficient of these graphite fluorides increases significantly. Only the samples having undergone post-heat treatment at 200° C. and at 300° C. retain a stable friction coefficient of 0.08 and 0.07, respectively, but this always remains higher than the friction coefficient obtained with the subfluorinated carbons of the invention, as may be observed in FIG. 3 and FIG. 4.

Thus, not only do the subfluorinated carbons used in the invention have very low friction coefficients compared to all the fluorinated carbons, carbon fluorides and graphites used in the prior art as solid lubricants, but they can also be used as solid lubricants under vacuum, under ultrahigh vacuum, in dry or humid air, or in a liquid or viscous dispersant such as an oil.

1. A solid lubricant comprising subfluorinated carbons simultaneously comprising fluorinated carbon domains with a (CF)n structure and nonfluorinated graphitic carbon domains, in powder form.

2. The solid lubricant as claimed in claim 1, characterized in that the nonfluorinated graphitic carbon domains have at least one dimension of between 1 nanometer and 1 micron inclusive.

3. The solid lubricant as claimed in claims 1, characterized in that the nonfluorinated graphitic carbon domains have at least one dimension of between 1 nanometer and 300 nanometers inclusive.

4. The solid lubricant as claimed in claim 1, characterized in that the molar percentage of graphitic carbon compared to the total number of moles of subfluorinated carbon is 5% or more but lower than 100%.

5. The solid lubricant as claimed in claim 1, characterized in that the subfluorinated carbon is obtained by fluorinating a carbon matrix at a temperature between 300° C. and 500° C. inclusive.

6. The solid lubricant as claimed in claim 5, characterized in that the carbon matrix has a graphitic structure.

7. The solid lubricant as claimed in claim 6, characterized in that the carbon matrix having a graphitic structure consists of nanofibers and/or nanotubes and/or nanocones and/or nanodisks and/or nanoparticles of graphitic carbon.

8. The solid lubricant as claimed in claim 5, characterized in that the carbon matrix consists of graphitic carbon nanofibers and in that said matrix is fluorinated by direct fluorination at a temperature between 370° C. and 500° C. inclusive.

9. The solid lubricant as claimed in claim 5, characterized in that the matrix consists of graphitic carbon nanofibers and is fluorinated by direct fluorination at a temperature between 400° C. and 425° C. inclusive.

10. The solid lubricant as claimed in claim 5, characterized in that the carbon matrix consists of carbon and/or coke and/or petroleum pitch having a graphitic structure.
